(19) United States
(12) Patent Application Publication
(10) Pub. No.: US 2010/0129310 A1
(43) Pub. Date: May 27, 2010

(54) STABILIZED DESLORATADINE COMPOSITION

(76) Inventors: Pavak Rajnikanth Mehta, Uttar Pradesh (IN); Indu Bushan, Hyderabad (IN); Vijay Dinanathji Nasare, Maharashtra (IN); Mailatur Sivaraman Mohan, Hyderabad (IN); Rahul Sudhakar Gawande, Hyderabad (IN); Ravinder Kodipyaka, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 11/573,505

(22) PCT Filed: Aug. 8, 2005
(86) PCT No.: PCT/US05/28009

§ 371 Date: Feb. 9, 2010

(51) Int. Cl. A61K 31/444; B01J 41/12

(52) U.S. Cl. 424/78.15; 521/32; 514/290

(57) ABSTRACT

Stabilized desloratadine-containing pharmaceutical compositions are prepared using complexes formed by combining desloratadine and a resin in the acidic form.

The present invention relates to a stabilized composition containing desloratadine or its salts. The invention further relates to a process of stabilizing desloratadine.

This invention relates to pharmaceutical compositions containing 8-chloro-6,11-dihydro-11-(4-piperidinylidene)-5H-benzo[5,6]-cyclohepta[1,2-b]pyridine (hereinafter desloratadine, “descarbonylethoxyloratadine,” or “DCL”) that resist the formation of desloratadine decomposition products, suitable for oral administration to treat allergic reactions in mammals.

D. D. Wirth et al., “Maillard Reaction of Lactose and Fluoxetine Hydrochloride, a Secondary Amine,” Journal of Pharmaceutical Science, Vol. 87(1), pages 31-39, 1998, has explained that drugs which are secondary amines (not just primary amines as had sometimes been reported) undergo the Maillard reaction with lactose under pharmaceutically relevant conditions. Hence there is a need to inhibit the reaction, and various methods to prevent this reaction were described.

A review of the structure of desloratadine reveals that desloratadine is a secondary amine, which makes it susceptible to the Maillard reaction. N-formyl DCL has been reported to be the major impurity of desloratadine that is observed as a result of the usual formulating procedures.

The disclosures in U.S. Pat. No. 6,100,274 and U.S. Published Application 2002/0123504 have shown that, under typical manufacturing and storage conditions, DCL is not stable and degrades in the presence of lactose, a compound commonly used as a filler in various pharmaceutical dosage forms, such as tablets, capsules or powders. Over time, the lactose and DCL react to form a colored product, and there is a high degree of DCL degradation. The intensity of the color is typically dependent on the amount of DCL present, the conditions of storage, such as humidity and temperature, as well as the length of storage time.

Lactose may react with DCL, degrading it to form an enamine. Such a reaction may also occur with other similar reactive excipients, such as other mono- or di-saccharides. Hence, stable pharmaceutical compositions of DCL, or a pharmaceutically acceptable salt thereof, in blended, granulated or compressed form, which are substantially free of reactive excipients are especially desirable.

Studies have also shown that in the absence of unbound water very little to no degradation occurs in DCL compositions that include lactose. While under typical packaging and storage conditions, DCL pharmaceutical composition dosage forms would be exposed to unbound water, e.g., in the form of humidity, there are known manufacturing and storage procedures by which exposure to unbound water and humidity can be reduced or eliminated.

Traditionally, when pharmaceutical compositions or formulations are prepared, the active ingredient or therapeutic agent (e.g., DCL) is milled and/or screened to decrease the particle size and/or narrow the particle size distribution. Most often, this is done in order to optimize various physicochemical characteristics of the formulation, such as dissolution, content uniformity, bioavailability of the active ingredient, and the like. However, the interaction between DCL and reactive excipients, such as lactose, may be affected by the surface area of the DCL particles in the pharmaceutical composition or formulation.

Among the several means for inhibiting or preventing the interaction between DCL and reactive excipients, such as lactose, in a pharmaceutical composition is to prevent DCL from coming into contact with any reactive excipients in the composition. One manner in which this may be achieved is to coat the DCL particles with an inert or non-reactive coating prior to formulation with reactive excipients. Preferably, the inert coating should not significantly influence the pharmacodynamic characteristics (e.g., time to onset of efficacy, and adsorption in vivo) of the composition. Suitable inert film-forming agents include, but are not limited to, cellulosics, such as methylcellulose, hydroxymethyl cellulose, carboxymethylcellulose, hydroxypropyl methylcellulose, hydroxypropyl cellulose, hydroxyethylcellulose, methylhydroxyethylcellulo-se and sodium carboxymethyl cellulose; vinyls, such as polyvinyl pyrrolidone; glycols, such as polyethylene glycols; acrylics, such as dimethylaminoethyl methacrylate-methacrylate acid ester copolymer, and ethylacrylate-methylacrylate copolymer; and other carbohydrate polymers, such as maltodextrins, and polydextrose. Preferably, the inert coating agent contains a hydrophilic film-forming agent, such as hydroxypropyl methylcellulose, so that absorption in vivo is not significantly delayed.

U.S. Pat. No. 6,100,274 teaches that descarbonylethoxyloratadine discolors and decomposes in the presence of acidic excipients disclosed in the art. This patent discloses that these problems are substantially solved when the use of acidic excipients is avoided and descarbonylethoxyloratadine is combined with a pharmaceutically acceptable carrier medium comprising a DCL-protective amount of a pharmaceutically acceptable basic salt. Thus, the disclosure is a pharmaceutical composition comprising an anti-allergic effective amount of descarbonylethoxyloratadine in a pharmaceutically acceptable carrier medium comprising a DCL-protective amount of a pharmaceutically acceptable basic salt.

U.S. Published Application 2002/0123504 discloses the various methods for improving the stability of compositions containing desloratadine. These include use of anhydrous desloratadine, increase of the particle size, non hygroscopic desloratadine containing formulations, powder coating or coating of the granules of desloratadine with a protective agent, and prevention of use of lactose or other reactive excipients, i.e., the excipients which are acidic in nature.

The above mentioned patents have made several attempts to stabilize desloratadine but each of the above mentioned methods of formulation of desloratadine have limited the formulator in the choices of excipients available to formulate a stable formulation of desloratadine, especially for rapid disintegration and dissolution.

Hence there remains a need for the stabilization of desloratadine but at the same time offering the flexibility to the formulator to use a wide range of excipients.

A tablet-shaped oral lyophilizate product being sold in Europe and elsewhere, using the trademark AERIUS, contains desloratadine and the excipients gelatin, mannitol, aspartame, citric acid, and polacrilin potassium, a dye, and a flavoring agent. As described in the European Public Assessment Report for the drug that is available at the European Medicines Agency's website (http://www.emea.eu.int/humandocs/Humans/EPAR/aerius/aerius.htm), the product is prepared by dissolving all of the components except polacrilin potassium, dispersing the polacrilin potassium in the solution, and lyophilizing. Desloratadine is said to be bound to the polacrilin potassium resin, with a resin to drug ratio of 3:1, to reduce the bitter taste of the drug.

Desloratadine-containing products are being sold in the United States of America, using the trademark CLARINEX. One of the products is an orally-disintegrating tablet called CLARINEX™ REDITABS™ from Schering Corporation, Kenilworth, N.J. USA, which (according to the published prescribing information) contains 5 mg of desloratadine, gelatin Type B NF, mannitol USP, aspartame NF, polarcrillin (sic) potassium NF, citric acid USP, red dye, and tutti frutti flavoring.

### SUMMARY OF THE INVENTION

An aspect of the invention includes a complex formed from desloratadine and an ion exchange resin in the acidic form.

Another aspect of the invention includes a process for preparing a pharmaceutical dosage form, comprising:

a) combining desloratadine with an ion-exchange resin in the acidic form;

b) separating a solid resin complex of desloratadine; and

c) combining the solid resin complex with one or more pharmaceutical excipients.

In the process, the complex can be formed by combining desloratadine and the resin in an aqueous medium, one embodiment of which is a lower alkanol that contains about 1 to about 25 volume percent of water.

The complexes of the invention stabilize desloratadine against degradation reactions with pharmaceutical excipients, such as lactose.

### DETAILED DESCRIPTION

The present invention relates to stable pharmaceutical compositions of desloratadine and its pharmaceutically acceptable salts or esters and process for preparation of the same. The novel pharmaceutical compositions offer flexibility for the formulator to choose from a wide range of excipients.

Surprisingly, a stable complex of DCL with resins that are copolymers of methacrylic acid, or styrene, with divinylbenzene, which are acidic, can be formed. The complex exhibits no significant degradation of desloratadine during storage under the usual conditions.

Moreover the above complexation with copolymers of methacrylic acid or styrene with divinylbenzene enables the formulator to choose from a wide range of pharmaceutically acceptable excipients to formulate a stabilized pharmaceutical composition of DCL. The complex thus formed is stable and does not undergo degradation.

The resin used can be either a cation exchange resin or an anion exchange resin. Ion exchange resins useful in the practice of the present invention include, but are not limited to, anionic resins such as: DUOLITE™ AP143/1093 (Cholestyramine Resin USP, a copolymer of styrene and divinylbenzene, with quaternary ammonium functionality) and cationic resins such as: AMBERLITE™ IRP64 (Polacrilex Resin, a porous “polacrilin” copolymer of methacrylic acid and divinylbenzene). AMBERLITE IRF-66(H), AMBERLITE™ IR-118 (H), AMBERLITE™ IR-120, AMBERLYST™ XN-1010, DUOLITE C-20, AMBERLYST 15, DUOLITE C-25D, DUOLITE ES-26 and related acidic ion-exchange resins are also useful. The DUOLITE™, AMBERLYST™, and AMBERLITE™ resins are available from Rohm and Haas Company, Philadelphia, Pa. U.S.A. Other suitable resins, such as DOWEX HCR-W (FORMERLY DOWEX 50W) and DOWEX MSC-1, can be obtained from other manufacturers. The useful resins have an acidic pH when dispersed in water, and this is considered to be an “acidic form” of the resin.

The term “pharmaceutical composition,” as used herein, means a combination comprising a safe and effective amount of the DCL active ingredient, in admixture with one or more pharmaceutically acceptable excipients.

The term “pharmaceutically acceptable excipient,” or simply “excipient,” as used herein, means any physiologically inert, pharmacologically inactive material, which is compatible with the physical and chemical characteristics of the particular DCL compound active ingredient selected for use. Pharmaceutically-acceptable excipients include, but are not limited to, polymers, plasticizers, fillers, binders, lubricants, glidants, disintegrants, solvents, co-solvents, buffer systems, surfactants, preservatives, sweetening agents, flavoring agents, pharmaceutical grade dyes or pigments, and viscosity agents.

Useful binders for pharmaceutical compositions such as tablets are exemplified by, but not limited to, acacia, tragacanth, hydroxypropylcellulose, pregelantinized starch, gelatin, povidone, ethylcellulose, hydroxypropylcellulose, hydroxypropylmethylcellulose, and methylcellulose. A particularly useful binder is povidone.

Examples of useful diluents, fillers, or glidants for pharmaceutical compositions are lactose, mannitol, sorbitol, magnesium stearate, stearic acid, talc, colloidal silicon dioxide, starch, sodium starch glycolate, crospovidone, croscarmelose sodium, and microcrystalline cellulose. The selection of a particular diluent is not restricted to the use of basic excipients. Hence the formulator is not bound with respect to the use of specific diluents and fillers.

Flavoring agents that are useful for compositions of this invention include those described in A. R. Gennaro et al., Eds., Remington: The Science and Practice of Pharmacy, 20th Edition, Lippincott, Williams & Wilkins, Baltimore, Md., U.S.A., pages 1018-1027, 2000, incorporated by reference herein. The pharmaceutical compositions suitable for use herein generally contain up to about 5% of flavoring agents.

Useful sweeteners include, but are not limited to, sucrose, glucose, saccharin, sorbitol, malt extract syrup, mannitol, and aspartame. A particularly useful sweetener is aspartame.

The compositions of the present invention are prepared according to methods known to those skilled in the art. Basically, the preparation procedure involves dissolving or suspending DCL in an aqueous medium, followed by combining the solution or suspension with an ion exchange resin in the acidic form to produce a drug/resin complex. The drug/resin complex is isolated and then usually dried under controlled temperature, such as at about 60° C., to produce a desired moisture content, such as less than about 10% by weight, then the drug/resin complex is blended with formulation components such as lactose, magnesium stearate, silicon dioxide, talc, microcrystalline cellulose, and/or other desired excipients. In some instances, it will be desired to granulate the mixture, using wet or dry granulation, and techniques for this procedure are well known in the art. The mixture is then further placed into capsules or compressed into tablets for formulations of DCL that release drug into the digestive system, or it is formulated as an orally disintegrating tablet.

The pH condition for forming the desloratadine-resin complex is not particularly critical, and a pH between about 3 and about 7 will typically be used. Suitable weight ratios of desloratadine to resin generally are about 1:0.2 to about 1:10, or about 1:1 to about 1:5. The aqueous medium for forming a complex sometimes will contain a buffering agent that helps to maintain a desired pH, and useful buffer systems include, but are not limited to, one or more of acetic, boric, carbonic, phosphoric, succinic, maleic, tartaric, citric, benzoic, lactic, glyceric, gluconic, glutaric, citric acid, and glutamic acid buffers. A particularly useful buffering agent is citric acid.

The efficiency of complex formation can be enhanced, as shown by higher concentrations of desloratadine in the complex, by using a water-containing lower alkanol as the aqueous medium. Suitable lower alkanols include the aliphatic alcohols having 1 to about 5 carbon atoms, either branched or unbranched, such as methanol, ethanol, isopropanol, n-propanol, n-butanol, isobutanol, and the like, including mixtures of any two or more lower alkanols in any proportions. The lower alkanol typically will contain about 1 to about 25 volume percent of water, or about 5 to about 15 volume percent of water, but other concentrations are also useful.

Optionally, the tablet, capsule, etc. can be coated with a polymer composition to improve the appearance, resist attack by stomach acids, or perform other functions. Such coatings are well known to those skilled in the art.

The invention will be further illustrated by the following examples, which provide details for certain specific aspects of the invention and are not to be construed as limiting the scope of the claimed invention.

### Example 1

A complex of desloratadine and a resin was prepared, as follows:

1) 50 grams of desloratadine was dispersed in 2500 grams of water;

2) Citric acid was added to the drug suspension of step 1) to produce a suspension pH of 6.5;

3) 100 grams of resin (AMBERLITE IRP64) was added to the mixture of step 2) and stirred for 1 hour;

4) the dispersion of step 3) was filtered and the complex obtained was dried at 60° C. until the loss on drying, as measured at 105° C., was <10% by weight;

5) to the filtrate of step 4), 50 grams of resin (AMBERLITE IRP64) was added and the dispersion was stirred for 1 hour;

6) the dispersion of step 5) was filtered and the complex obtained was dried at 60° C. until the loss on drying, as measured at 105° C., was <10% by weight; and

7) the dried complexes of steps 4) and 6) were combined.

### Example 2

A complex of desloratadine and a resin was prepared, as follows:

1) 50 grams of desloratadine was dispersed in 2500 grams of water;

2) Citric acid was added to the drug suspension of step 1) to produce a suspension pH of 6.5;

3) 50 grams of resin (AMBERLITE IRP64) was added to the mixture of step 2) and stirred for 1 hour;

4) the dispersion of step 3) was filtered and the complex obtained was dried at 60° C. until the loss on drying, as measured at 105° C., was <10% by weight;

5) to the filtrate of step 4), 50 grams of resin (AMBERLITE IRP64) was added and the dispersion was stirred for 1 hour;

6) the dispersion of step 5) was filtered and the complex obtained was dried at 60° C. until the loss on drying, as measured at 105° C., was <10% by weight;

7) to the filtrate of step 6), 50 grams of resin (AMBERLITE IRP64) was added and the dispersion was stirred for 1 hour;

8) the dispersion of step 7) was filtered and the complex obtained was dried at 60° C. until the loss on drying, as measured at 105° C., was <10% by weight; and

9) the dried complexes of steps 4), 6), and 8) were combined.

This combined complex was subjected to stability testing in a closed high density polyethylene bottle at 40° C. and 75% relative humidity for three months. The impurity profile of the complex, as determined by high performance liquid chromatography (“HPLC”) during the stability studies, was as shown in the table below, where each value is expressed in percent:

| Impurity | Initial | 1 month | 2 months | 3 months |
|---|---|---|---|---|
| Total | 0.059 | 0.0754 | 0.1089 | 0.1053 |
| N-Formyl DCL | ND | 0.0161 | 0.0399 | 0.0446 |

The above data show that the complex did not show a significant rise in impurity levels during the stability study

### Example 3

A complex of desloratadine and a resin was prepared, as follows:

1) 100 grams of AMBERLITE IRP64 was dispersed in 500 grams of water under high-speed stirring for 6 hours;

2) 50 grams of AMBERLITE IRP64 was dispersed in 250 grams of water under high speed stirring for 6 hours;

3) 50 grams of desloratadine was dispersed in 1250 grams of water under high speed stirring;

4) citric acid was added to the drug suspension of step 3 to produce a pH about 6.5;

5) to the drug suspension of step 4), the resin dispersion of step 1 was added and stirred for 1 hour;

6) the dispersion of step 5) was filtered and the complex was dried at 60° C. until the loss on drying, as measured at 105° C., was <10% by weight.

7) to the filtrate of step 6), the resin dispersion of step 2 was added and stirred for 1 hour;

8) the dispersion of step 7) was filtered and the complex was dried at 60° C. until the loss on drying, as measured at 105° C., was <10% by weight;

9) the dried complexes of step 6) and step 8) were combined.

### Example 4

A complex of desloratadine and a resin was prepared, as follows:

1) 50 grams of desloratadine were dispersed in 500 ml methanol, containing about 10% by volume of water, stirred for 5 to 10 minutes at 25-30° C., and filtered to obtain a clear solution;

2) 150 grams of AMBERLITE IRP64 resin were added and stirring was continued at the same temperature for about 2 hours;

3) the resin complex that formed was isolated by filtration, washed with about 100 ml of methanol containing about 10% by volume of water, dried under vacuum at ambient temperature for about 10-5 minutes, and finally dried under high vacuum at 60-65° C. for about 8 hours.

The resin complex contained 25.3% by weight desloratadine, as determined by HPLC.

### Example 5

Tablets containing desloratadine were prepared, using the following:

| Ingredient | mg/Tablet | |
|---|---|---|
| Desloratadine complex of Example 3 | 25.32 | |
| | (5 mg of desloratadine) | |
| Mannitol (PEARLITOL SD 200*) | 157.18 | |
| Crospovidone | 10 | |
| Aspartame | 18 | |
| Lactose | 25 | |
| Flavor | 1.25 | |
| Colloidal silicon dioxide | 2 | |
| Talc | 5 | |
| Sodium stearyl fumarate | 6.25 | |
| Total | 250 | mg |

*PEARLITOL is a trademark of Roquette Freres, Lestrem, France.

Desloratadine complex, crospovidone, aspartame and about one-half of the mannitol were sifted through a 40 mesh sieve. The remaining mannitol and the lactose were sifted together through a 40 mesh sieve. Combined sifted ingredients were loaded into a double cone blender and blended for 15 minutes.

Talc, colloidal silicon dioxide, and the flavor were sifted together through a 60 mesh sieve and added to the ingredients in the double cone blender and blending was continued for 5 minutes.

Sodium stearyl fumarate was sifted through a 60 mesh sieve, added to the ingredients in the double cone blender and blending continued for another 10 minutes, and the lubricated blend was compressed into tablets using a punch and die.

A bioequivalence study was conducted using the prepared tablets (hereinafter called “Test” or “T”) and the commercially available product CLARINEX™ REDITABS™, 5 mg desloratadine orally disintegrating tablets from Schering Corporation of Kenilworth, N.J. USA (hereinafter called “Reference” or “R”). The study was a single dose, randomized, two-way crossover study with a 14 days washout period between the products, carried out using 13 healthy adult subjects.

The results of the study are shown in the following table:

| | | | 90% Confidence |
|---|---|---|---|
| Attribute | Absolute values | T/R | interval of T/R |
| AUC₀₋ₜ | Test: 35135.56 pg · hr/mL | 100.66 | 87.89-115.28 |
| | Reference: 35120.6 pg · hr/mL | | |
| Cₘₐₓ | Test: 1773.56 pg/mL | 92.85 | 82.09-115.28 |
| | Reference: 1904.89 pg/mL | | |

The AUC₀₋ₜ value represents the area under the drug plasma concentration-time curve, beginning at the time of dosing and ending at the last measured concentration, and the Cₘₐₓ value is peak plasma concentration of the drug.

### Example 6

Desloratadine-containing tablets were prepared from the following:

| Ingredient | mg/Tablet | |
|---|---|---|
| Desloratadine complex of Example 1 | 32.9 | |
| | (5 mg of desloratadine) | |
| Mannitol (PEARLITOL SD 200) | 148.35 | |
| Crospovidone | 10 | |
| Aspartame | 18 | |
| Lactose | 25 | |
| Flavor | 2.5 | |
| Colloidal silicon dioxide | 2 | |
| Talc | 5 | |
| Sodium stearyl fumarate | 6.25 | |
| Total | 250 | mg |

Desloratadine complex, crospovidone, aspartame and a portion of the mannitol were sifted through a 40 mesh sieve.

Lactose and the remaining portion of mannitol were sifted together through a 40 mesh sieve.

The sifted ingredients were loaded into a double cone blender and blended for 15 minutes.

Talc, colloidal silicon dioxide, and the flavor were sifted together through a 60 mesh sieve, added to the ingredients in the double cone blender, and blended for 5 minutes.

Sodium stearyl fumarate was sifted through a 60 mesh sieve, added to the ingredients in the double cone blender, and the mixture was further blended for 10 minutes.

The lubricated blend was compressed into tablets using a punch and die.

The tablets were subjected to a stability study by storing in closed high density polyethylene bottles at 40° C. and 75% relative humidity for 3 months. The impurity profile of the tablets during the stability studies is shown in table below, where values are in area-percent as measured by HPLC:

| | | | 2 | 3 |
|---|---|---|---|---|
| Attribute | Initial | 1 month | months | months |
| Total Impurities | 0.0899 | 0.1568 | 0.1006 | 0.1289 |
| N-Formyl DCL | 0.0048 | 0.0163 | 0.0112 | 0.0106 |

The above data shows that the tablet formulation did not develop a significant rise in impurity levels during the stability study.

For comparison purposes, a similar stability study was carried out using the commercially available CLARINEX™ REDITABS™, 5 mg desloratadine orally disintegrating tablets from Schering Corporation, Kenilworth, N.J. USA. The impurity profile of the commercial product during the stability studies is shown in the table below:

| Attribute | Initial | 2 months | 3 months |
|---|---|---|---|
| Total Impurities | 0.1911 | 0.2618 | 0.4825 |
| N-Formyl DCL | 0.0342 | 0.0926 | 0.1026 |

### Example 7

Compatibility of desloratadine with various excipient materials was determined by mixing the drug and an excipient, then subjecting the mixture to direct exposure for 14 days at 40° C. and 75% relative humidity. The analytical results for the presence of N-formyl desloratadine in the samples (as determined by HPLC) were as tabulated below, where ratios are expressed on a weight basis. Desloratadine and polacrilin resin (AMBERLITE IRP64) were dispersed in water and stirred for 2 hours, isolated by filtration, and then dried at 60° C. until the loss on drying was not more than 10% w/w when tested using an infrared moisture balance at 105° C. Where citric acid was used, the quantity of citric acid was selected to adjust the pH of the dispersion to about 6.5. Where lactose was used, the quantity of lactose is 6 times the weight of other materials, and the lactose is dry mixed with the desloratadine or dried desloratadine-resin mixture.

| | % N-Formyl DCL | | |
|---|---|---|---|
| | | After | |
| Sample Description | Initial | exposure | Comments |
| Desloratadine + Polacrilin | Nil | Nil | DCL stable with acidic |
| (1:2) | | | excipient |
| Desloratadine + Polacrilin | Nil | Nil | DCL stable with acidic |
| (1:3) | | | excipient |
| Desloratadine + Polacrilin | Nil | Nil | DCL stable with acidic |
| (1:2) + Citric acid | | | excipient |
| Desloratadine | Nil | Nil | — |
| Desloratadine + Polacrilin | Nil | 0.02% | Minimal rise in impurity |
| (1:2) + Lactose | | | level |
| Desloratadine + Polacrilin | Nil | 0.02% | Minimal rise in impurity |
| (1:3) + Lactose | | | level |
| Desloratadine + Polacrilin | Nil | 0.01% | Minimal rise in impurity |
| (1:2) + Citric acid + | | | level |
| Lactose | | | |
| Desloratadine + Lactose | Nil | 0.08% | Significant increase in |
| | | | impurity |

A review of the above data indicates that desloratadine is stable with polacralin resin, which is acidic in nature. N-formyl-desloratadine is a degradation product of desloratadine. The art suggests that it is the major degradant of desloratadine, which is produced in the presence of an acidic excipient. But a review of the above data reveals that when DCL is complexed with polacrilin resin, DCL does not undergo any degradation to form N-formyl DCL for up to two weeks at accelerated conditions of 40° C. and 75% RH. This shows the stability of DCL in the presence of acidic excipients.

Moreover, the art discloses the need for lactose-free formulations and the effect of lactose on the increase in the degradation of N-formyl DCL. But the above data reveal that the combination of desloratadine, polacrilin (1:2) and lactose shows only marginal rise of the degradation product N-formyl DCL with respect to the initial concentration. On the contrary, desloratadine and lactose shows a high concentration of the degradation product N-formyl DCL after exposure to 40° C. and 75% RH for two weeks.

Hence, the above data reveal that the invention is a useful method of stabilizing desloratadine in formulations comprising acidic excipients. The said invention also is a new dimension in formulating a stable formulation of desloratadine whereby the formulator is not bound by limitations in selecting a suitable excipient. The said invention offers the formulator the flexibility of selecting excipients from a wide range of available excipients.

### Example 8

An immediate-release tablet was prepared from the following ingredients:

| Ingredient | mg/Tablet |
|---|---|
| Desloratadine | 5 |
| AMBERLITE IRP64 | 10 |
| Lactose anhydrous | 38.5 |
| Microcrystalline cellulose | 80 |
| Pregelatinized starch | 7.5 |
| Crospovidone | 6 |
| Talc | 1.5 |
| Magnesium stearate | 1.5 |
| Total Weight | 150 |

Drug and resin were dispersed in water to prepare the complex, then filtered, dried, and mixed with the other ingredients, followed by compressing the mixture to make the tablet.

### Example 9

An orally disintegrating tablet was prepared using the following ingredients:

| Ingredient | mg/Tablet |
|---|---|
| Desloratadine | 5 |
| AMBERLITE IRP64 | 10 |
| Lactose anhydrous | 31.3 |
| Microcrystalline cellulose | 30 |
| Pregelatinized starch | 7.5 |
| Crospovidone | 9 |
| Mannitol | 50 |
| Aspartame | 3 |
| Peppermint flavor | 1.2 |
| Talc | 1.5 |
| Magnesium stearate | 1.5 |
| Total Weight | 150 |

Drug and resin were dispersed in water to prepare the complex, then isolated by filtration and dried. The remaining ingredients were added and mixed, followed by compression to make the tablet.

1. A complex formed from desloratadine and an ion exchange resin in the acidic form.

2. The complex of claim 1, wherein the resin comprises cholestryamine resin.

3. The complex of claim 1, wherein the resin comprises a polacrilin resin.

4. The complex of claim 1, having a weight ratio of desloratadine to resin about 1:0.2 to about 1:10.

5. The complex of claim 1, having a weight ratio of desloratadine to resin about 1:1 to about 1:5.

6. A pharmaceutical composition, comprising the complex of claim 1 and one or more excipients.

7. A process for preparing a pharmaceutical dosage form, comprising:
a) combining desloratadine with an ion-exchange resin in the acidic form;
b) separating a solid resin complex of desloratadine; and
c) combining the solid resin complex with one or more pharmaceutical excipients.

8. The process of claim 7, wherein desloratadine and a resin are combined in an aqueous medium.

9. The process of claim 8, wherein the aqueous medium has an acidic pH.

10. The process of claim 8, wherein the aqueous medium comprises a buffering agent.

11. The process of claim 8, wherein the aqueous medium comprises at least one of acetic, boric, carbonic, phosphoric, succinic, maleic, tartaric, citric, benzoic, lactic, glyceric, gluconic, glutaric, citric acid, and glutamic acids, as a buffering agent.

12. The process of claim 8, wherein the aqueous medium comprises a lower alkanol.

13. The process of claim 8, wherein the aqueous medium comprises a lower alkanol containing about 1 to about 25 percent by volume of water.

14. The process of claim 8, wherein the aqueous medium comprises a lower alkanol containing about 5 to about 15 percent by volume of water.

15. The process of claim 8, wherein the aqueous medium comprises methanol.

16. The process of claim 7, wherein a pharmaceutical excipient comprises lactose.
